(19) United States
(12) Patent Application Publication
(10) Pub. No.: US 2023/0036913 A1
(43) Pub. Date: Feb. 2, 2023

(54) TERAHERTZ WAVE PLETHYSMOGRAPHY

(71) Applicants: Yu Rong, Tempe, AZ (US); Panagiotis Theofanopoulos, Tempe, AZ (US); Georgios Trichopoulos, Tempe, AZ (US); Daniel W. Bliss, Phoenix, AZ (US)

(72) Inventors: Yu Rong, Tempe, AZ (US); Panagiotis Theofanopoulos, Tempe, AZ (US); Georgios Trichopoulos, Tempe, AZ (US); Daniel W. Bliss, Phoenix, AZ (US)

(73) Assignee: Arizona Board of Regents on behalf of Arizona State University, Scottsdale, AZ (US)

(21) Appl. No.: 17/865,841

(22) Filed: Jul. 15, 2022

Related U.S. Application Data
(60) Provisional application No. 63/222,664, filed on Jul. 16, 2021.

(51) Int. Cl. A61B 5/1455; A61B 5/00; A61B 5/01; A61B 5/024; A61B 5/0295; A61B 5/08; G16H 40/67

(52) U.S. Cl. CPC A61B 5/14551; A61B 5/0017; A61B 5/0077; A61B 5/01; A61B 5/02416; A61B 5/0295; A61B 5/0816; A61B 5/7278; G16H 40/67

(57) ABSTRACT

Terahertz wave plethysmography provides a new principle of radar-based vital sign detection. This disclosure presents new applications at terahertz (THz) frequency band for non-contact cardiac sensing. For the first time, cardiac pulse information is shown to be simultaneously extracted based on two established principles using unique THz waves. A novel concept of Terahertz-Wave-Plethysmography (TPG) is introduced, which detects blood volume changes in the upper dermis tissue layer by measuring the reflectance of THz waves, similar to the existing remote photoplethysmography (rPPG) principle. A detailed analysis of pulse measurement using THz is provided. The TPG principle is justified by scientific deduction and carefully designed experimental demonstrations. Additionally, pulse measurements from various peripheral body regions of interest (ROIs), including palm, inner elbow, temple, fingertip, and forehead, are demonstrated using a novel ultra-wideband (UWB) THz sensing system.

### PRIORITY CLAIM

This application is a non-provisional conversion of, and claims the benefit of priority to U.S. Provisional Application Ser. No.: 63/222,664 filed Jul. 16, 2021 entitled “TERAHERTZ WAVE PLETHYSMOGRAPHY”, the disclosure of which is incorporated herein by reference in its entirety.

### FIELD OF THE DISCLOSURE

The present disclosure relates to non-contact sensing of vital signs, and in particular to terahertz (THz) radar-based vital sign sensing.

### BACKGROUND

Microwave (1 gigahertz (GHz) to 60 GHz) radars are widely used for detection of human vital signs, such as heart rate (HR), breathing rate (BR) and body temperature, which are biometrics for healthcare development. Explicitly, these systems leverage advanced signal processing techniques such as complex signal demodulation and phase-based methods to extract vital signs from the captured backscattered signals. These methods were initially developed for narrowband and later extended to wideband radars offering better clutter performance. However, the robustness of these systems is hindered by certain limitations arising due to the low operation frequency and limited radio frequency (RF) resources in these bands.

Currently developed Doppler signal processing techniques have difficultly providing accurate pulse measurements when a dynamic breathing pattern is present, not to mention the presence of other sources of random body motion artifacts. The fractional bandwidth (BW) of these frequencies leads to low-range-bin resolution, which results in increased cluttering noise, especially in crowded environments/targets. For example, a 15% BW at 60 GHz is 9 GHz, leading to 1.7 centimeters (cm) range resolution. However, the heart motions on the body surface are less than 1 millimeter (mm), hence the signal of the cardiac pulses is hard to detect within such large range-bins that include breathing motions (a few mm to 1 cm) and other body micro-motions, along with cluttering noise from other scatterers (e.g., clothing).

In addition, the small apertures needed for mobile applications or embedded systems lead to wide beams that capture the backscattered signals of multiple targets, further increasing cluttering noise. Even though solutions to these issues have been explored with active motion and sensor fusion techniques, these configurations remain limited in complex target scenes where multiple scatterers are located within the field-of-view (FOV).

### SUMMARY

Terahertz wave plethysmography provides a new principle of radar-based vital sign detection. This disclosure presents new applications at terahertz (THz) frequency band for non-contact cardiac sensing. For the first time, cardiac pulse information is shown to be simultaneously extracted based on two established principles using unique THz waves. The first fundamental principle is micro-Doppler (mD) motion effect, initially introduced in a coherent laser radar system and first experimentally demonstrated for vital sign detection. This motion-based method, primarily using coherent phase information from the radar receiver, has been widely exploited in microwave frequency bands and has recently found popularity in millimeter waves (mmW).

The second fundamental principle is reflectance-based optical measurement using infrared or visible light. The variation in the light reflection is proportional to the volumetric change of the heart, often referred to as photoplethysmography (PPG). PPG has been a popular technology for pulse diagnosis. Recently it has been widely incorporated into various smart wearables for long-term monitoring, such as fitness training and sleep monitoring. A high-level review on non-contact cardiac sensing is provided and it summarizes the prior works from microwave all the way to visible light with methodologies explained.

A novel concept of Terahertz-Wave-Plethysmography (TPG) is introduced, which detects blood volume changes in the upper dermis tissue layer by measuring the reflectance of THz waves, similar to the existing remote PPG (rPPG) principle. A detailed analysis of pulse measurement using THz is provided. The TPG principle is justified by scientific deduction and carefully designed experimental demonstrations. Additionally, pulse measurements from various peripheral regions of interest (ROIs), including palm, inner elbow, temple, fingertip, and forehead, are demonstrated using a novel ultra-wideband (UWB) THz sensing system.

Among the ROIs under test, it is found that the measurements from the forehead ROI gives the best accuracy with mean heart rate (HR) estimation error 1.51 beats per minute (BPM) and standard deviation (std) 1.08 BPM. The results validate the feasibility of radar based TPG principle for direct pulse monitoring. Last but not least, pulse sensitivity dependence of skin types is investigated and demonstrated for TPG measurements, which is a known result for non-contact reflectance PPG.

An exemplary embodiment provides a method for non-contact vital sign measurement of a subject. The method includes receiving a THz radar return signal measuring a region of interest of the subject; processing the radar return signal to jointly produce micro-Doppler data and reflectance-based data of the region of interest; and estimating vital sign information of the subject from the micro-Doppler data and the reflectance-based data.

Another exemplary embodiment provides a TPG sensor. The TPG sensor includes a THz radar sensor; and a signal processor configured to: receive a radar return signal from the THz radar sensor; measure a skin reflectance of the radar return signal; and extract vital sign information of one or more subjects based on the skin reflectance.

Another exemplary embodiment provides a non-transitory computer-readable medium comprising computer-readable instructions, that in response to being executed by a processor, cause the processor to receive a radar return signal from a THz radar sensor. The instructions also cause the processor to measure a skin reflectance of the radar return signal. The instructions also cause the processor to extract vital sign information of one or more subjects based on the skin reflectance.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1 is a schematic diagram of an exemplary three-dimensional (3D) terahertz (THz) radar system for enhanced imaging and vital sign extraction in a cluttered environment.

FIG. 2 is a schematic diagram of another 3D THz radar system for enhanced imaging and vital sign extraction in a cluttered environment.

FIG. 3 is a graphical representation of an example electromagnetic (EM) wave spectra of interest in embodiments described herein.

FIG. 4A is a schematic diagram of vital sign motion extraction using through-wall radar.

FIG. 4B is a schematic diagram of the principle of human vital sign measurement using micro-Doppler.

FIG. 5 is a graphical representation of wavelength dependence of the AC/DC ratio of a reflectance contact photoplethysmography (PPG) signal.

FIG. 6A is a schematic diagram of remote PPG (rPPG) signal extraction from videos.

FIG. 6B is a schematic diagram illustrating the rPPG principle with skin tissue.

FIG. 7 is a schematic diagram of a THz-wave-plethysmography (TPG) system for sensing vital signs.

FIG. 8 is a schematic diagram of upper skin structure during a cardiac cycle.

FIG. 9A is a graphical representation of the simulated skin reflection coefficient for different dermis conductivity values in the 285-315 gigahertz (GHz) spectrum.

FIG. 9B is a graphical representation of the THz measurement data versus time.

FIG. 10 is an image of a representative THz measurement setup in which the reference signals PPG and rPPG are acquired simultaneously.

FIG. 11 is a graphical representation of a comparison of raw waveforms from different measurement sensor outputs and their associated spectra.

FIG. 12 is a graphical representation of spectrograms of the different measurement data.

FIG. 13 illustrates the TPG measurement setups at various peripheral body regions of interest (ROIs).

FIG. 14 is a graphical representation of heart rate (HR) estimation error histograms at the various peripheral body ROIs of FIG. 13.

FIG. 15 is a graphical representation of a stepped-frequency continuous-wave (SFCW) radar transmission scheme used in embodiments described herein.

FIG. 16 is an exemplary method for performing Terahertz-Wave-Plethysmography according to one or more embodiments disclosed herein.

FIG. 17 is a block diagram of a TPG sensor or other system or device suitable for implementing non-skin contact vital sign measurement of a subject according to embodiments disclosed herein.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Terahertz wave plethysmography provides a new principle of radar-based vital sign detection. This disclosure presents new applications at terahertz (THz) frequency band for non-contact cardiac sensing. For the first time, cardiac pulse information is shown to be simultaneously extracted based on two established principles using unique THz waves. The first fundamental principle is micro-Doppler (mD) motion effect, initially introduced in a coherent laser radar system and first experimentally demonstrated for vital sign detection. This motion-based method, primarily using coherent phase information from the radar receiver, has been widely exploited in microwave frequency bands and has recently found popularity in millimeter waves (mmW).

The second fundamental principle is reflectance-based optical measurement using infrared or visible light. The variation in the light reflection is proportional to the volumetric change of the heart, often referred as photoplethysmography (PPG). PPG has been a popular technology for pulse diagnosis. Recently it has been widely incorporated into various smart wearables for long-term monitoring, such as fitness training and sleep monitoring. A high-level review on non-contact cardiac sensing is provided and it summarizes the prior works from microwave all the way to visible light with methodologies explained.

A novel concept of Terahertz-Wave-Plethysmography (TPG) is introduced, which detects blood volume changes in the upper dermis tissue layer by measuring the reflectance of THz waves, similar to the existing remote PPG (rPPG) principle. A detailed analysis of pulse measurement using THz is provided. The TPG principle is justified by scientific deduction and carefully designed experimental demonstrations. Additionally, pulse measurements from various peripheral regions of interest (ROIs), including palm, inner elbow, temple, fingertip, and forehead, are demonstrated using a novel ultra-wideband (UWB) THz sensing system.

Among the ROIs under test, it is found that the measurements from the forehead ROI gives the best accuracy with mean heart rate (HR) estimation error 1.51 beats per minute (BPM) and standard deviation (std) 1.08 BPM. The results validate the feasibility of radar based TPG principle for direct pulse monitoring. Last but not least, pulse sensitivity dependence of skin types is investigated and demonstrated for TPG measurements, which is a known result for non-contact reflectance PPG.

### I. Introduction

The use of higher-operating frequencies such as millimeter wave (mmW) and THz (100 GHz-10 THz) can potentially alleviate the limitations of microwave radar systems described above. Namely, these non-ionizing frequencies offer high fractional bandwidth that allow for increased range resolution and thus less cluttering noise. For instance, a 15% BW at 300 GHz is 45 GHz, resulting in a 3.3 mm range resolution. In addition, small physical apertures are electrically larger in these frequencies (compared to microwaves) due to the small wavelength, leading to narrow beams that can be focused only on one target/person, thus further decreasing cluttering noise from undesired scatterers. Finally, the small motions caused by pulse-related surface skin motion and blood flow on/near the body surface can be easily detected with phase-based methods, since the phase sensitivity increases due to the smaller wavelength. For example, a 0.5 mm motion causes a phase change of 3.6 degrees at 60 GHz, while the respective phase change at 300 GHz is 18 degrees. This allows for the detection of heart-caused micro-motions even in peripheral body sites based on mD motion effect. Breathing-interference-free pulse measurement is possible at these sites because they are further away from the upper torso body area.

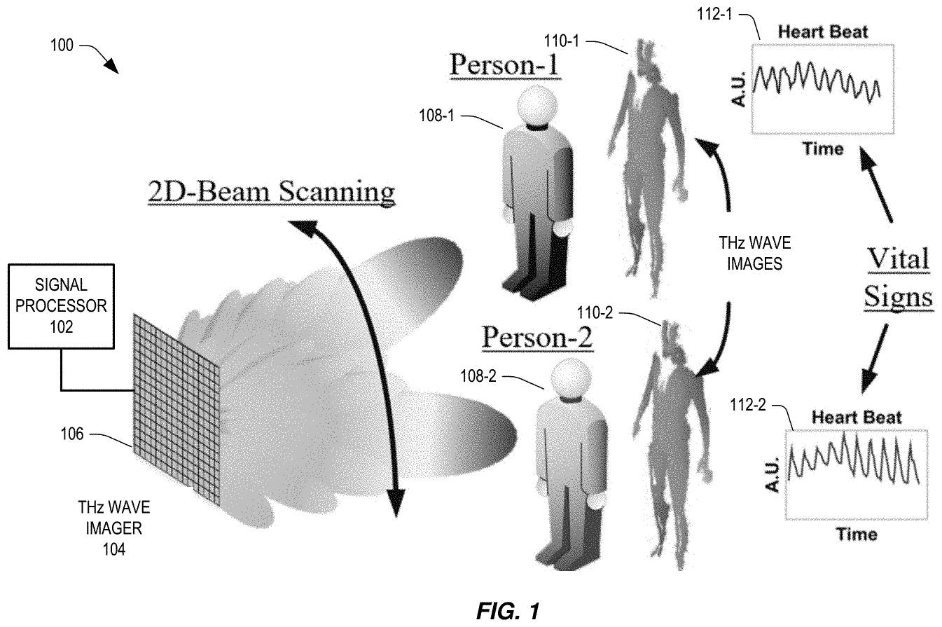

FIG. 1 is a schematic diagram of an exemplary three-dimensional (3D) THz radar system 100 for enhanced imaging and vital sign extraction in a cluttered environment. The THz radar system 100 can include a THz wave antenna array 106 that transmits THz waves. A THz wave imager 104 can receive radar return signals that are reflected off the skin or dermis layer of the subjects 108-1 and 108-2. A signal processor 102 can analyze the radar returns to determine THz wave images 110-1 and 110-2 that correspond to the subjects 108-1 and 108-2. By analyzing the images or other radar return signal data, the signal processor 102 can determine vital signs 112-1 and 112-2 associated with the subjects 108-1 and 108-2 respectively, where the vital signs 112-1 and 112-2 can be biometric information capable of being determined via TPG, such as a heart rate, a heartbeat waveform, a heart rate variability (HRV), vascular aging information, or artery stiffness information. It is to be appreciated that while FIG. 1 depicts a plurality of subjects, in other embodiments, the THz radar system 100 can determine vital signs or biometric parameters from a single person, or can determine vital signs or biometric parameters from a plurality of regions of interest in one or more subjects.

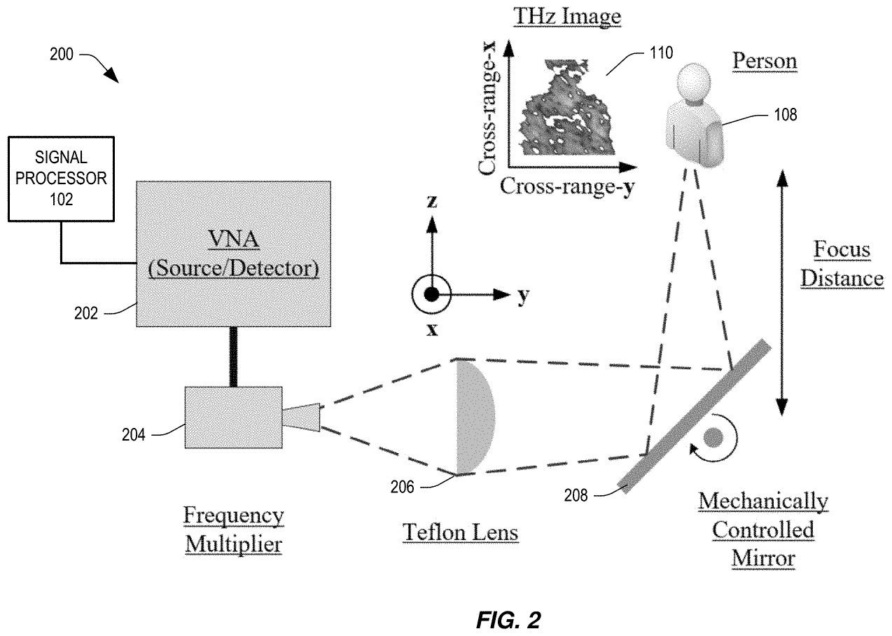

FIG. 2 is a schematic diagram of another 3D THz radar system 200 for enhanced imaging and vital sign extraction in a cluttered environment. In combination with the aforementioned, these higher-operating frequencies are also exploited for high spatial resolution 3D imaging (both range and 2D cross-range) due to their directive beams and large fractional BW. Thus, as shown in FIGS. 1 and 2, physically small systems can be deployed that form 3D images of various targets. Then, by analyzing the captured data by a signal processor 102, the vital signs of each person within the field-of-view (FOV) can be identified. Moreover, these systems can be leveraged to focus the beam on different parts of the human body detecting pulse information from multiple body parts offering new opportunities, including blood circulation inspection and remote blood pressure measurements from a distance, even through clothes.

In FIG. 2, provides additional detail about the THz wave imager 104 of the 3D THz radar system 200. The THz wave imager 104 can include a Vector Network Analyzer (VNA) Source/Detector 202, a frequency multiplier 204, a Teflon lens 206, and a mechanically or electromechanically controlled mirror 208 that is able to direct the radar transmission to a region of interest on a subject 108 in order to determine one or more biometric parameters of the subject 108 from the resulting THz images 110.

In this disclosure, an UWB sub-THz system is developed that creates narrow beams focusing the waves on different parts of the body and extracts the vital signs using the proposed TPG concept based on the reflectivity changes in the magnitude response, rather than the mD phase. Example body parts are evaluated, including forehead, temple, inner elbow, palm, and fingertip. In this manner, the high-resolution THz images can be exploited to strategically choose the body parts that can offer distortionless vital sign sensing. More importantly, evaluation results indicate that further valuable information from the pulse signal (in addition to simple heart rate) can be extracted for non-contact cardiovascular health assessment, such as vascular aging and artery stiffness.

### II. Remote Cardiac Sensing from Microwave to Visible Light

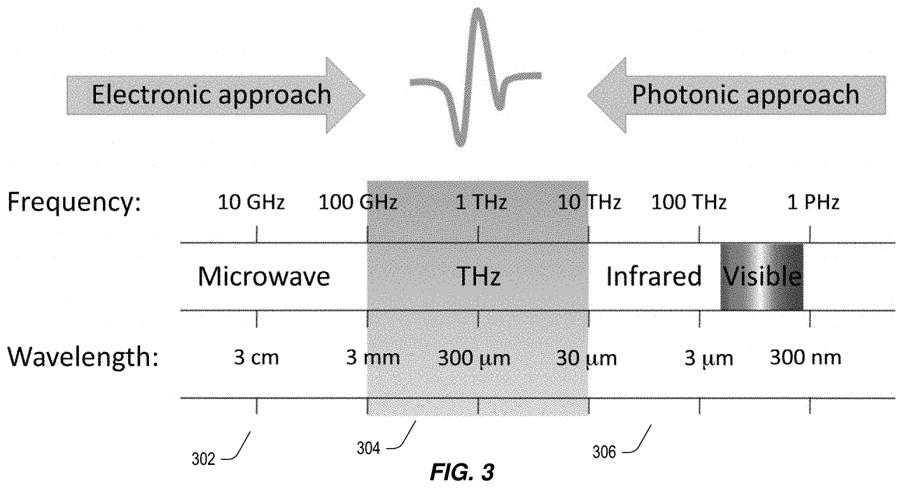

FIG. 3 is a graphical representation of an example electromagnetic (EM) wave spectra of interest in embodiments described herein. The spectral location of THz waves implies unique EM properties. The THz band sits between the microwave band and the visible light spectrum, and thus inherently possess the optical-like features from the visible light, and also the valuable phase information as seen in microwave band. Physiological measurements using each of these frequency bands, ranging from microwave (10⁹ Hz), mmW (10¹⁰ Hz), THz (10¹¹ Hz), infrared (10¹² Hz) and visible light (10¹⁴ Hz), have been reported previously.

Non-contact vital sign monitoring (VSM) technologies revolutionize the diagnosis experience among patients and medical professionals. It offers a convenient and comfortable way to take HR and BR measurements over a long period of time since it does not require direct physical contact. This could lead to many useful health monitoring applications, such as at residences, in elderly care, and infant monitoring. Various remote sensing devices are developed to demonstrate the possibility to detect cardiac pulse from a distance. The EM spectra of interest ranges from microwave, a few gigahertz (10⁹ Hz), to visible light, about 1 petahertz (10¹⁵ Hz), and the corresponding wavelength is from a few centimeters to about 300 nanometers. These bands consist of non-ionizing radiations and thus can be used for biomedical applications.

The EM spectra in FIG. 3 can be divided into three portions. The long-wavelength region 302 (low frequency) covers microwave and mmW; the short-wavelength region 306 (high frequency) includes infrared and visible light. In between, THz radiation 304 exhibits both photon-like and electron-like properties, which is demonstrated in this disclosure for pulse detection. Studies of the long-wavelength region rely predominantly on electronics, whereas studies of the short-wavelength region rely predominantly on photonics, resulting in a gap between these two research fields because of the limited availability of effective THz-generating sources and THz-sensitive detectors. But recently with the rapid development of THz science and technology, extensive research of biological effects of THz radiation has been conducted in the field of life science.

A. Micro-Doppler Motion

Two physical principles have been previously established for non-contact physiological measurement. First, in the long-wavelength region, microwave and mmW sensors were shown to detect human heartbeat based on the well-known mD principle. In 1975, a microwave technique was first proposed for measuring respiratory movements of humans and animals. Later, an X-band life detection radar was developed for detecting heartbeat and breathing of human subjects. More recently, mmW multiple-input multiple-output sensors were shown to detect heartbeats from multiple subjects due to improved spatial degree of freedom and phase signal sensitivity.

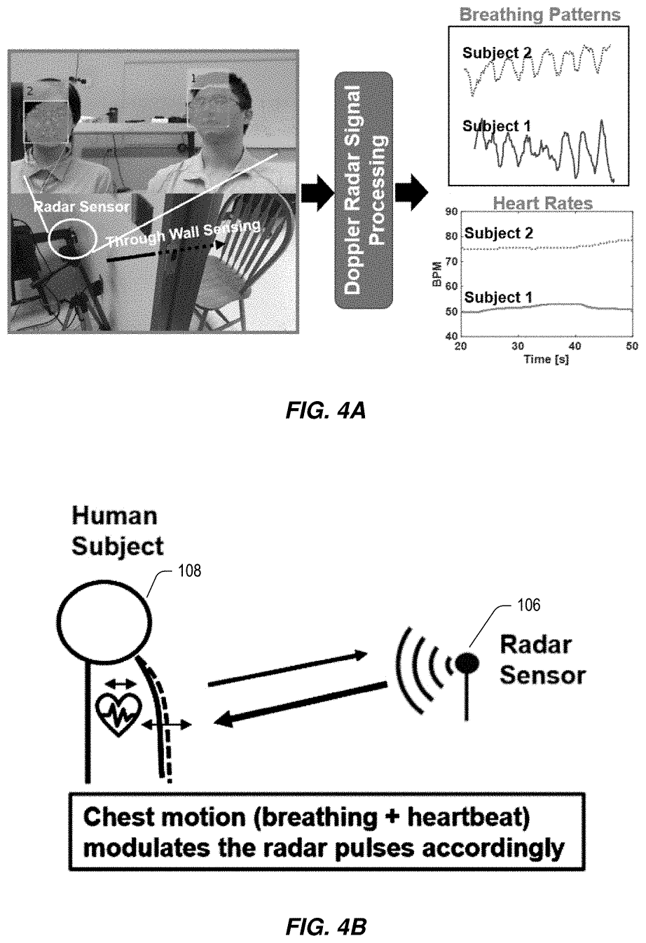

FIGS. 4A and 4 B illustrate an operational example of a mD bio-radar system. FIG. 4A is a schematic diagram of vital sign motion extraction using through-wall radar. FIG. 4B is a schematic diagram of the principle of human vital sign measurement using mD. The transmit antenna 106 sends a train of radar pulses towards the human subject 108. These pulses are reflected by the moving chest wall. The periodic expansion and contraction of the chest movement generates an observable change known as mD shift that is acquired by the receiving antenna. By processing the radar return, the vital sign signals are extracted. Often phase information is used for vital sign detection because of the simple linear relation between the phase rotation and the skin/tissue/body motion under test driven by cardiovascular activity depending on the operating frequency.

$$ϕ = \frac{4 π {x}_{h} ( t )}{λ} \qquad Equation 1$$

where xₕ(t) denotes the heartbeat motion and λ denotes the wavelength.

The major challenge for accurate HR detection using mD motion methods with microwave narrowband systems is motion coupling, that is the mixing effect of multitude stronger breathing signal and weak pulse signal of interest due to the aforementioned small BW and large FOV. In real situations, the precise pulse wave reconstruction is not possible except in some high signal-to-noise ratio regions. For long-term monitoring applications, random and involuntary body motion is inevitable. Practical and robust methods for motion-tolerant pulse detection using long-wavelength signals remain an open question.

B. Photoplethysmography

Second, in the short-wavelength region, photoplethysmography (PPG) is another non-invasive physical principle for monitoring pulse/HR. Similar to the gold standard for HR monitoring electrocardiography (ECG), PPG requires direct physical contact with the subject. The popularity of the PPG technology as an alternative HR monitoring technique has recently increased, mainly due to the simplicity of its operation, the wearing comfortability for its users, and its cost effectiveness. The operation of reflectance-mode PPG requires two components, a light source and a photodetector. PPG relies on measuring the pulse-like variations in light absorption in an illuminated skin area caused by the difference in absorption curves of oxygenated and deoxygenated blood. The amplitude of the variations depends on the amount of blood rushing into the peripheral vascular bed, the optical absorption of blood, skin pigmentation, ambient light, and the wavelength used to illuminate the blood.

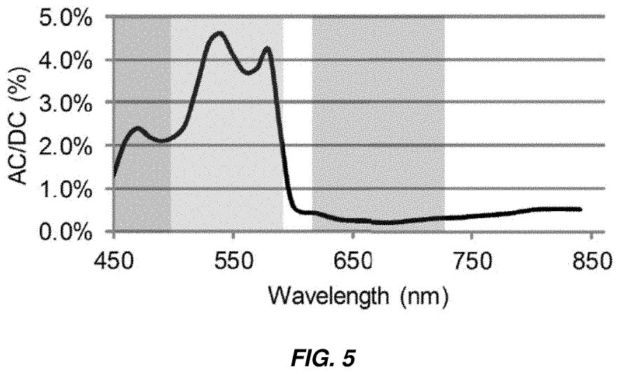

FIG. 5 is a graphical representation of wavelength dependence of the AC/DC ratio of a reflectance contact PPG signal. Here, DC denotes the pulse baseline and AC the pulsatile amplitude. Different optical wavelengths (e.g., infrared, green) interact differently with blood and tissues, involving several physical processes, i.e., scattering, absorption, reflection, transmission and fluorescence. In reflectance-mode, green light is one of the most commonly used colors because it contains more pulsatile information compared with other colors, according to FIG. 5.

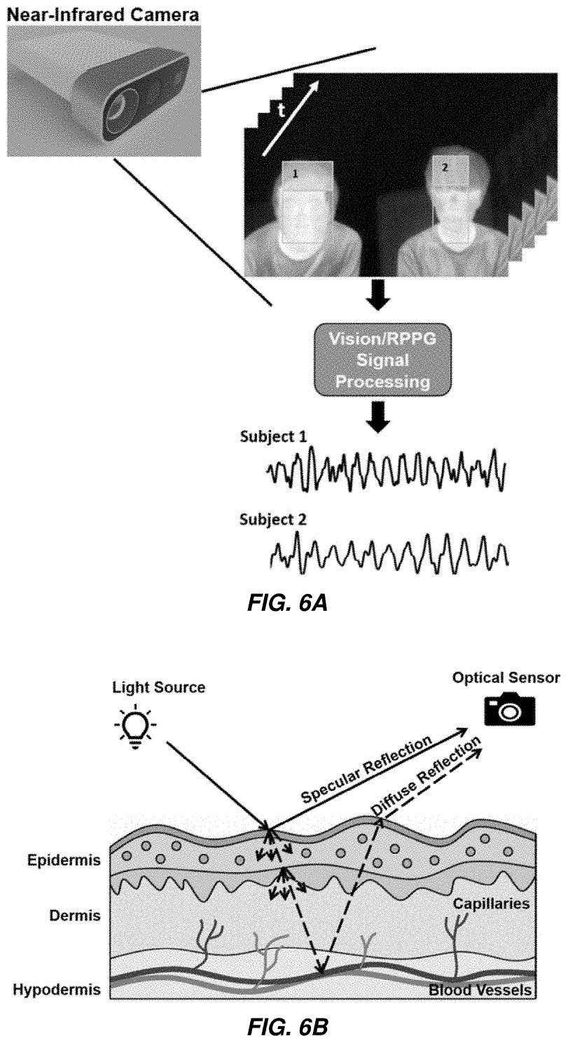

FIGS. 6A and 6B illustrate the working principle of an example remote PPG (rPPG). FIG. 6A is a schematic diagram of rPPG signal extraction from videos. FIG. 6B is a schematic diagram illustrating the rPPG principle with skin tissue. The reflectance PPG principle also motivated the use of digital cameras to measure plethysmographic signals from face or naked skin videos under ambient light conditions. This technique is often referred as remote PPG (rPPG) or imaging PPG. Recently infrared cameras have been included in rPPG for heartbeat detection due to their resilience to low-light and variable-light conditions.

C. Comparison of VSM in EM Waves

The EM spectra for vital sign measurement (VSM) approaches can be broadly divided into four categories, microwave (including mmW), THz, infrared, and visible light. Due to the aforementioned reasons, there is less effort on VSM using THz. Before presenting the new insights on using THz for VSM, it is worthwhile to review the current advances of mD bio-radar sensors and rPPG optical sensors.

rPPG using optical sensors (infrared and visible light) are advantageous over microwave bio-radars for motion tolerant VSM. That is because rPPG signal is from reflectivity change (or skin color shift) not from motion. High resolution images from optical sensors provides abundant information for signal processing. Other body motion artifacts can be separated via computer vision techniques by leveraging millions of image pixels and multiple color channels. Infrared is heavily investigated due to privacy issues of using normal color cameras. In general, optical sensors do not penetrate materials like clothes and blankets. They are also limited in line-of-sight (LOS) applications.

On the other hand, conventional narrowband mD radars with limited array size (mostly single antenna systems) are not able to handle realistic dynamic motion profile. The popular Doppler phase signal is more susceptible to chest motion and other random body movements, which are stronger than the pulse signal in the radar return due to larger radar-cross-section and physical displacement. Direct pulse measurement from the chest area is not possible without respiration suppressed and naïve spectral separation is not sufficient for HR estimation due to breathing motion and heartbeat coupling effects. Active motion cancellation techniques consider UWB, dual-radars, and RF front end re-design producing encouraging results, but their effectiveness can be further explored.

A THz system is shown for pulse detection at peripheral body sites because of excellent phase sensitivity due to smaller wavelength. Breathing-free pulse measurement is achievable at major peripheral artery sites, such as the wrist, with large BW and focusing beam at THz. Furthermore, this disclosure goes one step further and demonstrates measurable plethysmographic signals at face and other body parts in the THz magnitude response. Therefore, this measurement is named Terahertz-Wave-Plethysmography (TPG). A detailed comparison of VSM using EM waves is tabulated in Table 1.

TABLE 1
Summary of features of EM frequencies for vital sign detection
VSM Using EM Waves

| | | | | |
|---|---|---|---|---|
| Technology | Microwave (a few | THz (100- | Near- | Visible Light |
| | to tens of GHz) | 1000 GHz) | Infrared/ | |
| | | | Infrared | |
| Signal Type | Magnitude, phase, | Magnitude, phase | Magnitude only | Magnitude only |
| | or complex | | | |
| Working Principle | Motion based | Wave reflectivity/ | Mostly light | Mostly light |
| | Doppler effect | motion based | reflectivity | reflectivity |
| | | Doppler effect | | |
| Algorithm | Low phase | Good phase | Good pulse | Better pulse |
| Performance | sensitivity | sensitivity | sensitivity | performance from |
| | | | | more color channels |
| Vital Signs | Breathing dominant/ | Breathing and | Heartbeat detectable/ | Heartbeat detectable/ |
| Detection | heartbeat not robust | heartbeat separable | breathing not robust | breathing not robust |
| Computation Load | Low | Low | High | High |
| Spatial | Low (large | Good millimeter | Excellent | Excellent |
| Resolution | aperture) | resolution | | |
| Clutter | Poor | Intermediate | Good | Good |
| Performance | | | | |
| Penetration | Excellent | Good | Poor | Non-existent |
| Losses (e.g., | Low | Intermediate | High | Infinite |
| through smoke) | | | | |
| Synergy with | Poor | Excellent | None | None |
| Radar Imaging | | | | |
| (e.g., NLOS) | | | | |
| Cost and | Cheap | Very high | High | Very cheap |
| Manufacturing | | | | |
| Effort | | | | |
| Privacy Issue | None | None | Mild | Yes |

### III. Terahertz-Wave-Plethysmography (TPG)

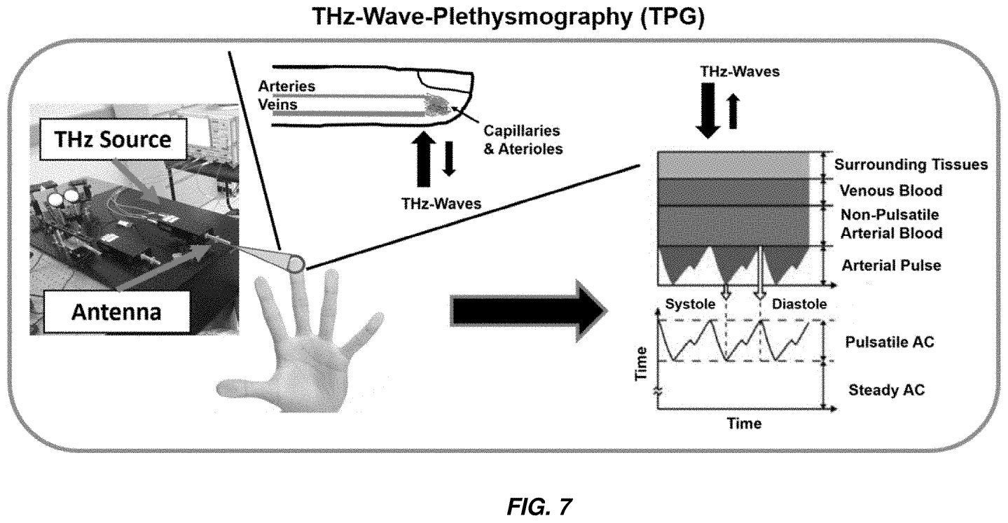

FIG. 7 is a schematic diagram of a THz-wave-plethysmography (TPG) system for sensing vital signs. The novel concept of TPG, which detects blood volume changes in the dermis layer by measuring the reflectance of THz wave, is similar to the PPG principle. THz waves can reach the dermis layer throughout the peripheral body parts. Similar skin optical properties found in near-infrared and visible light for plethysmography are also found in THz waves, such that THz interacts with hemoglobin in blood cells. There are measurable differences in the spectra of blood and its components when the hemoglobin content changes in the THz frequencies. It therefore can be inferred that the pulsatile variation exists in the THz wave absorption in an illuminated skin area caused by the difference in absorption curves of oxygenated and deoxygenated blood, and thus TPG is possible. In the following, full wave EM simulation and human subject experimental results are presented to validate the proposed theory.

A. Full-Wave Simulation Study

The acquired THz measurements are compared with theoretical data validating the use of reflectivity for the extraction of pulse. The reflectivity-based process is commonly used in the optical spectrum, where an infrared emitter or ambient light illuminates the skin and the intensity of the backscattered lights are modulated. Using the time-variant magnitude response of the reflected signals, the HR can be extracted.

This modulation in reflectivity has a twofold cause. Firstly, the amount of blood present in the subcutaneous skin vessels and capillaries changes leading to more blood (thus more losses) in the reflected waves. The second cause of this modulation is blood consistency. Namely, the amount of oxygen in the blood varies within the cardiac cycle and the losses of the EM waves are proportional to this variation. For example, the sensitivity of green light radiation to the oxygen levels in the blood is well established enabling the use of green light sensors for the detection of pulse. However, it was demonstrated using THz spectroscopy that waves ranging from 0.1-1 THz are also sensitive to the consistency of blood. Thus, it is assumed that the magnitude modulation in the measurements is attributed to the sub-skin conductivity variation, caused both by the amount of blood in the subcutaneous capillaries and the oxygen concentration in it.

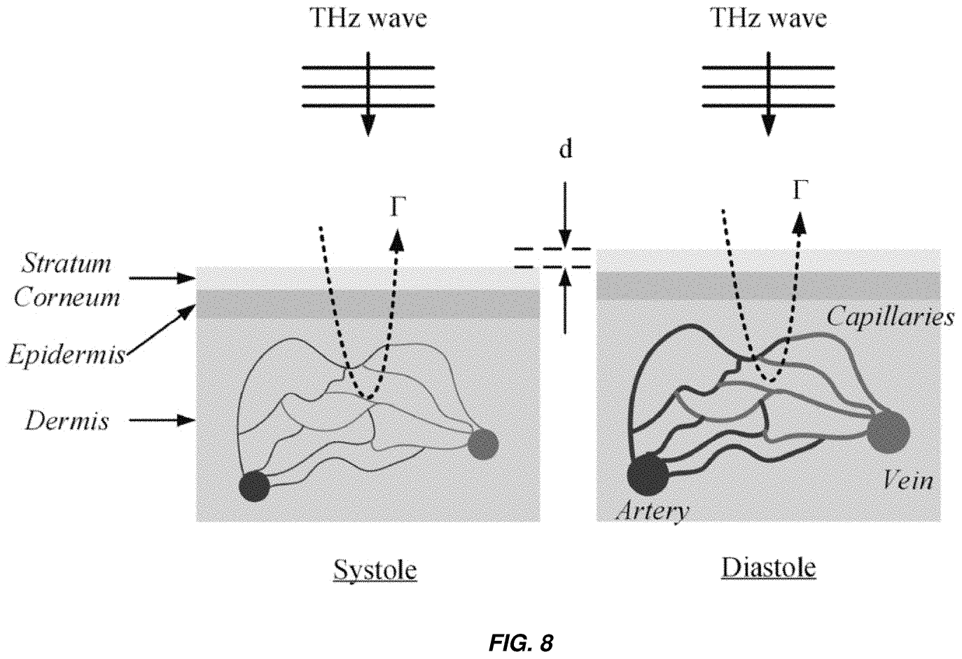

FIG. 8 is a schematic diagram of upper skin structure during a cardiac cycle. To study the reflectivity modulation caused by the cardiovascular activity at the peripheral body sites, the skin model shown in FIG. 8 is considered. The skin is modeled as a three-layered structure: the top part is the thin layer of the stratum corneum, followed by the epidermis where the presence of capillaries is very limited, and finally the dermis which is modeled as a semi-infinite layer. In the systole phase, the blood is lower leading to lower conductivity. On the contrary, during the diastole phase, the arteries and capillaries expand, leading to greater blood concentration, thus higher conductivity.

The EM material properties of these layers are tabulated in Table 2. As such, the stratum corneum and the epidermis have low conductivity and the dermis is a conductive layer since it is filled with capillaries and veins. During the cardiac cycle, it is assumed that the conductivity of the dermis is modulated, leading to the modulation of the reflected THz waves.

TABLE 2
Skin Model Parameters

| | | | σ_{diastole} | σ_{systole} |
|---|---|---|---|---|
| | Thickness | ηr | (S/m) | (S/m) |
| Stratum Corneum (sc) | 5 | 2.4 | 10⁻⁵ | 10⁻⁵ |
| Epidermis (ep) | 90 | 3.2 | 1 | 1 |
| Dermis (derm) | Indefinite- | 3.9 | 41 | 36 |
| | half-space | | | |

The dermis layer conductivity as given in Table 2, is calculated by,

σ_{dermis}=(1−BC)σ_{skin,dry}+BCσ_{blood} Equation 2

where a σ_{skin,dry} is the dry skin conductivity, σ_{blood} is the blood conductivity, and BC is the blood concentration.

In an embodiment, the blood concentration is 60% during the systole and 70% during the diastole. The reflection coefficient of the skin is given by,

$${Γ}_{skin} = \frac{{r}_{1} + {r}_{2} {z}_{1} + {r}_{1} {r}_{2} {r}_{3} {z}_{2} + {r}_{3} {z}_{1} {z}_{2}}{1 + {r}_{1} {r}_{2} {z}_{1} + {r}_{2} {r}_{3} {z}_{2} + {r}_{1} {r}_{3} {z}_{1} {z}_{2}} \qquad Equation 3$$

where

$${r}_{1} = \frac{{η}_{sc} - {η}_{air}}{{η}_{sc} + {η}_{air}} \qquad Equation 4$$

$${r}_{2} = \frac{{η}_{ep} - {η}_{sc}}{{η}_{ep} + {η}_{sc}} \qquad Equation 5$$

$${r}_{3} = \frac{{η}_{derm} - {η}_{ep}}{{η}_{derm} + {η}_{sc}} \qquad Equation 6$$

$$and$$

$${z}_{1} = {e}^{- 2 {ik}_{sc} {t}_{sc}} \qquad Equation 7$$

$${z}_{2} = {e}^{- 2 {ik}_{ep} {t}_{ep}} \qquad Equation 8$$

where

$${η}_{air} = \sqrt{{ε}_{0}} \qquad Equation 9$$

$${η}_{x} = \sqrt{{ε}_{x} ( 1 - i \frac{{σ}_{x}}{2 π f {ε}_{x}} )} \qquad Equation 10$$

$$and$$

$${k}_{x} = \frac{2 π f \sqrt{{ε}_{x}}}{c} , x = sc , ep , or derm \qquad Equation 11$$

where the subscript χ denotes either the stratum corneum, the epidermis, or the dermis, ƒ the frequency, ε₀ the free space permittivity, and c the speed of light in free space.

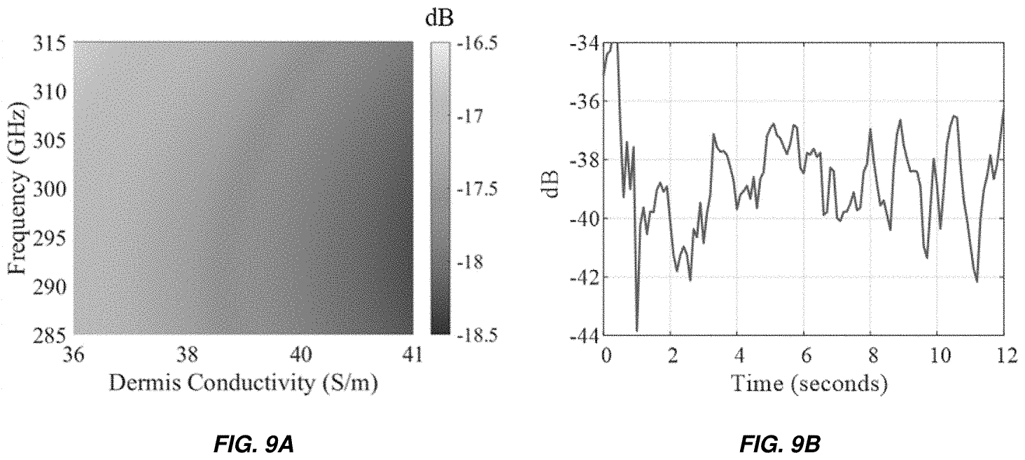

FIG. 9A is a graphical representation of the simulated skin reflection coefficient for different dermis conductivity values in the 285-315 GHz spectrum. FIG. 9B is a graphical representation of the THz measurement data versus time. Using the above-mentioned equations, the magnitude of the skin reflection coefficient is calculated in the 300-330 GHz range for various values of σ_{dermis}. As such, the peak-to-peak modulation expected on the THz reflection coefficient during the cardiac cycle is close to 1.5 dB, which is in agreement with the actual THz measurements presented in FIG. 9B. Therefore, using this theoretical approach the effect of the reflectivity change measured using the proposed THz setup is associated to the blood concentration in the upper layers of the skin during the cardiac cycle. This effect enables pulse detection using THz reflectivity measurements, which are not dominated by the breathing motions and other body motions, thus, leading to a robust pulse monitoring tool.

### IV. Results

A. TPG Measurements

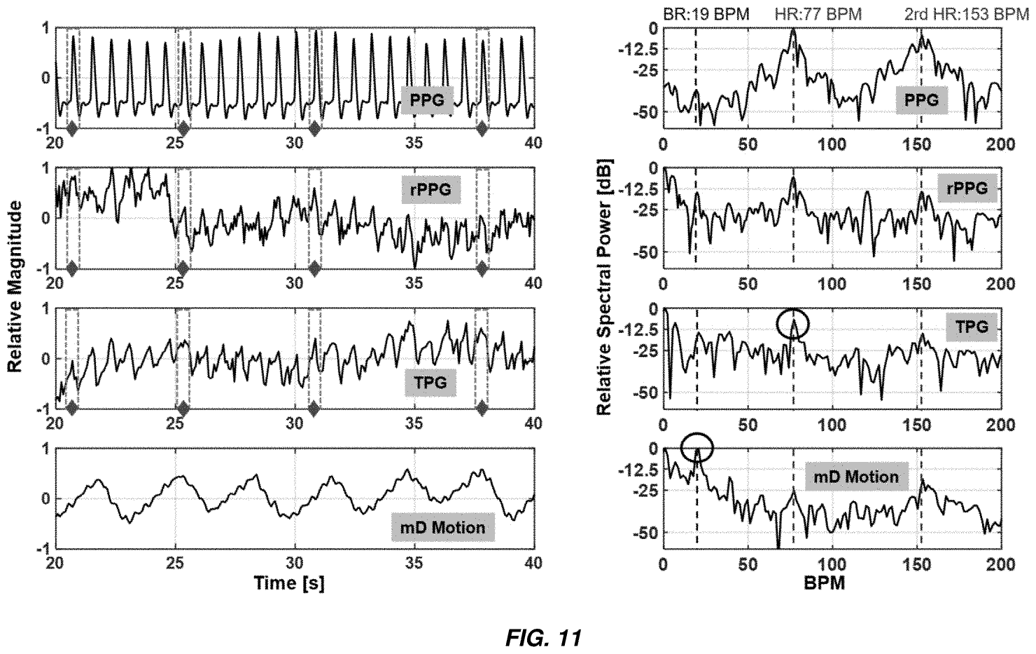

TPG measurements are THz reflectance measurement from magnitude response. Simultaneously TPG and mD motion, measurement from phase, are extracted using the proposed UWB THz system. Representative TPG and mD motion measurements along with multiple reference measurements are illustrated in FIG. 11. In this example, the forehead of a test subject is illuminated by the THz sensing system. It can be concluded that the magnitude variation mostly corresponds to the THz reflectivity change, similar to the PPG principle. On the other hand, the extracted phase variation of the THz return is related to the macro body motion, such as physical movements, breathing motion, and it captures the skin surface vibration, inner tissue movement at the top dermis layer due to pulsation and slight arm movement due to breathing activity. Physiological motion is a quasi-periodic narrowband signal. The fundamental frequency signatures such HR and BR change slowly over time. Therefore, in a short processing window, HR and BR can be estimated by inspecting the peak spectral energy location at the proper frequency regions.

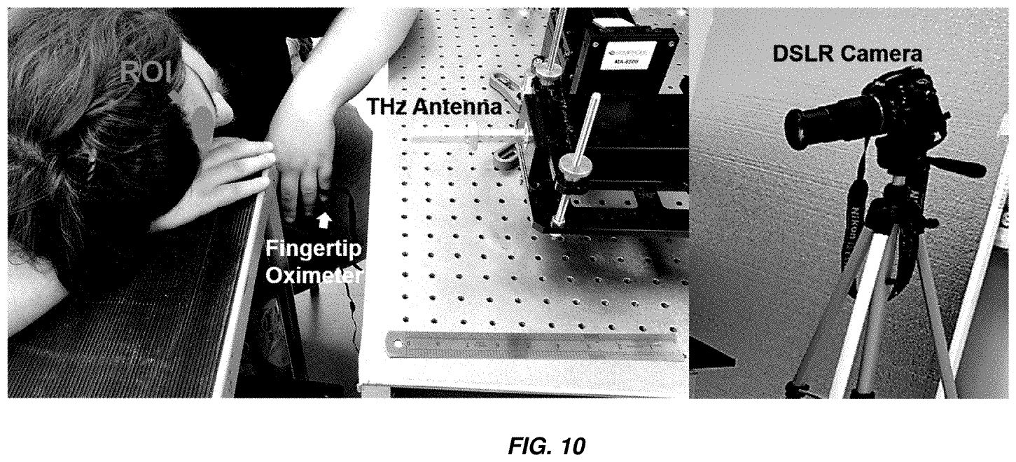

FIG. 10 is an image of a representative THz measurement setup in which the reference signals PPG and rPPG are acquired simultaneously. Typical measurement setup at a forehead region of interest (ROI) is indicated. A fingertip oximeter is used for providing a contact PPG signal for comparison. Simultaneously, a digital single-lens reflex (DSLR) camera Nikon D750 is focused on the forehead area and recording at 30 frame per second with 1920×1080 pixel resolution. The rPPG signal is extracted by processing the sequence of images. To avoid any man-made artifact and maintain purity of the signal components, the raw magnitude and phase information are used to demonstrate the advantages of TPG measurements.

FIG. 11 is a graphical representation of a comparison of raw waveforms from different measurement sensor outputs and their associated spectra. The left column represents, from top to bottom, the PPG waveform, the rPPG waveform, the TPG waveform and finally the mD phase waveform. Minimum processing is applied on each sensor output (scaling) for visualization. The four different types of measurements are aligned. It is clear that pulsation signal is the dominant trend in PPG, rPPG and TPG, which intuitively makes sense because they are all reflectivity-based measurements. The diamond markers indicate aligned individual pulses in PPG, rPPG and TPG. While the mD phase is motion sensitive and is breathing-motion dominant. This observation is consistent with microwave and mmW radars for VSM.

The corresponding vital sign spectra are shown in the right column of FIG. 11. No filtering is applied for generating the spectra and only a Hanning window is used before Fourier transform. Similarly, the major spectral components in PPG, rPPG and TPG are fundamental HR and the 2nd-order harmonics of HR. Except strong DC component, the dominant spectral energy in mD phase is breathing rate (BR).

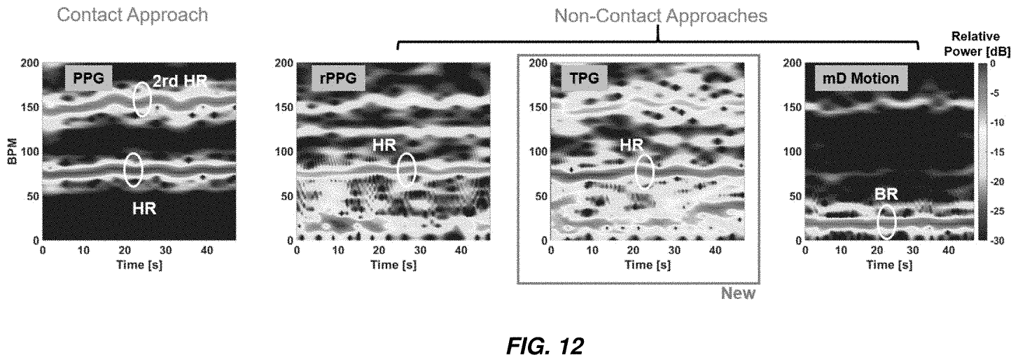

FIG. 12 is a graphical representation of spectrograms of the different measurement data. The time-frequency analysis is applied to the same dataset, with the spectrograms being generated using a short-time Fourier transform with a sliding window 13-second and one sample increment. The y-axis is the frequency in beats per minute (BPM). An infinite impulse response high pass filter with cut-off frequency 0.1 Hz is applied in the y-axis direction to remove the DC to emphasize the spectral energies of interest. From the left to the right, they are spectrograms of PPG, rPPG, TPG and mD phase. Based on the operation mode, they are divided into two categories: contact approach, PPG, and non-contact approaches including rPPG, TPG and mD phase. PPG, rPPG and TPG show similar spectral structures, in which the fundamental heartbeat and second-order harmonic of heartbeat are clearly visible and highlighted. Motion artifacts show up in rPPG and TPG in the form of lower frequency interference close to DC due to random body movement and breathing but they are not dominant. By inspecting the TPG and the mD motion results, it validates the underline principle of the novel TPG measurement such that TPG is mostly from reflectance measurement.

Overall, the contact approach PPG gives best performance, which is used as the standard pulse reference but it requires direct physical contact neutralizing the motivation of remote sensing. On the other hand, the results from the three different non-contact methods provide distinct implications. TPG is similar to the rPPG as the pulse signal almost maintains the stronger spectral components during the experiment. Compared to PPG, rPPG and TPG experience some low frequency interference due to involuntary body motion and breathing motion. These motion artifacts are not constant and dominant thus can be easily separated through post-processing. The mD phase measurement is known for motion sensitivity and captures breathing motion consistently during the experiment. In FIG. 11 second column last row, the highlighted breathing component is about 25 dB stronger than the possible fundamental pulse component and thus makes it challenging for robust pulse measurement, which is still an open question in microwave radar VSM.

Several observations can be made based on these carefully designed experiments. The breathing signal is not present at the TPG measurement and it validates that the magnitude change mostly from the THz reflectivity change. Plethysmography using THz wave, therefore, is feasible. This study helps demystify the origin of non-contact reflectance plethysmography. So far, the community has not reached consensus on the physical principles of rPPG. At least two hypotheses on the causes of the observed phenomenon are: 1) optical density change within the tissue caused by arterial pulsations and 2) local deformation of tissue caused by capillaries. Or stated differently, one is EM wave reflectivity change and the other one is local micro-tissue motion. The results confirm that the major contribution for non-contact reflectance plethysmography is EM wave reflectivity change. That is because the local micro-tissue motion is a much smaller physical displacement compared to the body motion related to respiratory activity, which in this particular example shows up in the phase based measurement. If the local tissue motion is the leading cause of the detected pulse in the magnitude response, then there has to be a stronger breathing component in TPG waveform and spectrum since it is much stronger than the local tissue motion.

B. Accuracy

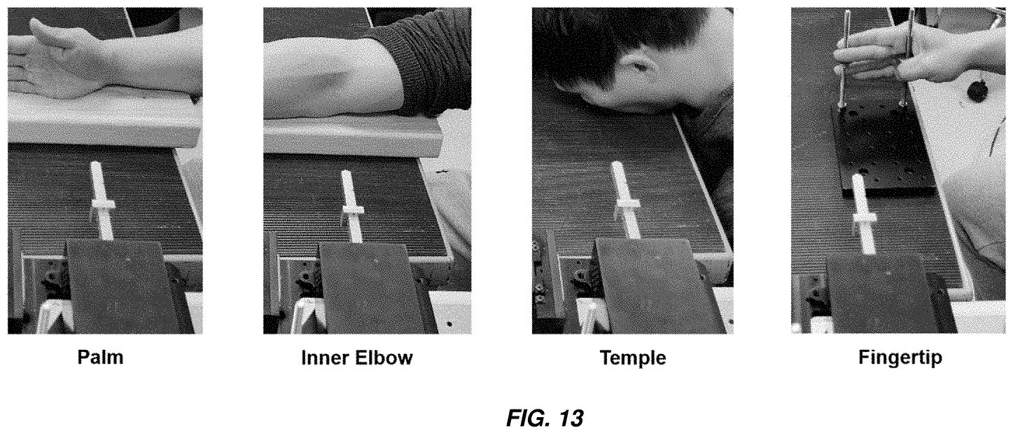

FIG. 13 illustrates the TPG measurement setups at various peripheral body ROIs. The accuracy of TPG measurements is demonstrated at four exemplary body sites: palm, inner elbow, temple and fingertip. These experiments were performed at Arizona State University Terahertz Electronics Lab. During the experiment, the test subjects were instructed to breath normally and maintain stationary in a relaxing state. However, random body motion and involuntary movements were observed during data acquisition and, in reality, they are inevitable especially when the experiment time increases.

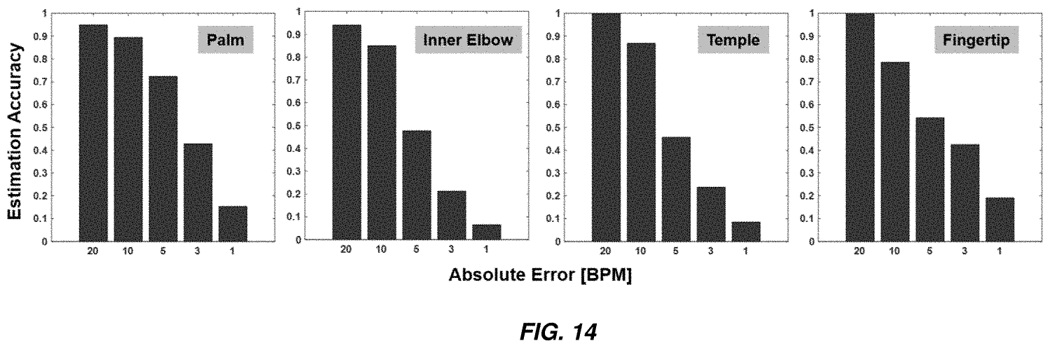

FIG. 14 is a graphical representation of HR estimation error histograms at the various peripheral body ROIs of FIG. 13. Four 240-second datasets are used for HR error analysis. The results in FIG. 14 are generated with a sliding window of 13-second with one sample increment. The HR estimation error histogram displays the error distribution at four different levels.

The HR estimation performance is calculated as the percentage of measurement points that its estimation error within (≤) 20, 10, 5, 3 and 1 BPM respectively. At palm, the measurement error distribution is 94.93%≤20, 89.38% ≤10, 72.39%≤5, 42.81%≤3, 15.36%≤1; inner elbow: 94.12%≤20, 84.97%≤10, 47.71%≤5, 21.24%≤3, 6.54%≤1; temple: 99.84%≤20, 86.76%≤10, 45.59%≤5, 23.69%≤3, 8.50%≤1; fingertip: 100%≤20, 78.51%≤10, 54.30% ≤5, 42.42%≤3, 19.12%≤1. Overall, on average the error statistics at palm, inner elbow, temple, fingertip and forehead are summarized in Table 3. HR estimation accuracy from the forehead is superior to the other four body ROIs because of the larger surface area at the forehead and better upper body stabilization in prone position as shown in FIG. 10. These results together validate the feasibility of radar based TPG principle for direct pulse monitoring.

TABLE 3
HR Estimation Performance at Peripheral Body Sites

| | Palm | Inner Elbow | Temple | Fingertip | Forehead |
|---|---|---|---|---|---|
| Mean (BPM) | 5.24 | 7.05 | 5.84 | 5.49 | 1.51 |
| std (BPM) | 6.91 | 6.69 | 3.71 | 4.78 | 1.08 |

### V. Discussion

The feasibility of radar based plethysmography was investigated using THz waves. Contact electrocardiac activity measurement devices, such as ECG and contact PPG measurement devices, are the gold standard to measure pulse/HR. The emerging remote sensing technologies using radar and vision sensors change the way of measuring a range of vital signs of the human body. A comprehensive review of operating principles and experimental results of the two exciting technologies was presented. For non-disturbance, ubiquitousness, all-weather, penetrability, and privacy-preserving sensing requirements, the radar technology is favored in these respects. A novel concept of TPG is proposed that exacts pulse information using the known optical principle PPG.

The implication that a THz radar system detects cardiac pulse based on photoplethysmography principle in addition to the mD principle was investigated by a multiplicity of measurements. Exemplary validation measurements show high similarities between radar TPG signal and reference contact-PPG signal regarding the R-peak locations and the spectral peak location. The presented comparison between radar TPG, and mD motion, rPPG, PPG proves the feasibility of radar based plethysmography detection. The analysis considered the differences regarding measurement principles, sizes of the measurement spots, and ROIs. Increased HR estimation error is observed at some ROIs. It is caused by the lower signal-to-noise-ratio (SNR) of the TPG, which can be explained by the surface curvature and area of the measurement spots and measurement stabilization, and which is substantiated by the higher variations of HR estimates. The TPG HR estimation performance can be enhanced by system-level optimization, such as improvement of the dynamic range and emission power of the utilized radar system, and processing optimization.

Furthermore, the new insight of cardiac physiology of THz waves interaction with human body at various ROIs improves the direct pulse monitoring performance in a non-contact fashion. The conventional mD approach focuses on the chest area. It generates noisy and inaccurate signal that is highly distorted by stronger body movement and breathing motion. Direct pulse monitoring and instantaneous inspection are not feasible using conventional approaches. Recently, research and technology in the field of THz science and electronics has undergone tremendous development, for example THz human body imaging. Being able to use high spatial resolution THz images to strategically detect pulse information, through clothing or bedding, from multiple spots of the human body opens new opportunities for biomedical applications using THz waves: inspecting blood circulation, extracting blood pressure related biometrics such as blood pulse pressure and pulse wave velocity. The unique features of THz waves, such that they exhibit electron-like and photon-like properties, implies two different ways of VSM. For the first time, radar technology is proven to be able to detect pulse signal using optical principle.

### VI. Methods

A. Terahertz Sensing System

The UWB THz sensing configuration is depicted in FIG. 2 and consists of a vector network analyzer (VNA) 202 that feeds a high frequency module 204 that up converts the signal in the 220-500 GHz range. Then, a highly directive horn antenna (206,208) radiates the THz waves towards the ROI of a human subject.

B. Radar Processing

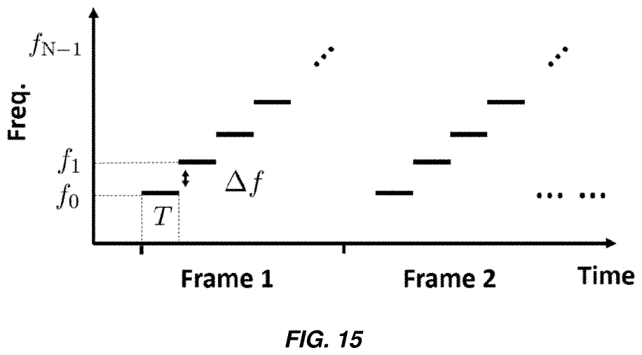

FIG. 15 is a graphical representation of a stepped-frequency continuous-wave (SFCW) radar transmission scheme used in embodiments described herein. The system uses a SFCW radar, which is an alternative architecture of the UWB radar system and operates in the frequency domain rather than time domain. The SFCW radar transmits a series of discrete narrow band pulses stepwise to achieve a larger effective bandwidth. As such, the modulated waveform consists of a group of N coherent pulses with pulse duration T, whose frequencies are ƒₙ=ƒ₀+nΔƒ. Assume that each SFCW waveform has N pulses called one SFCW frame and the center frequency of the first pulse is ƒ₀, as illustrated in FIG. 15.

One transmitted SFCW frame is represented as a sum of N windowed narrow band signals,

$${x}_{tx} ( τ ) = \frac{1}{\sqrt{T}} {\sum }_{n} ( \frac{τ - nT}{T} ) {e}^{j 2 π ( {f}_{0} + n Δ f ) τ} \qquad Equation 12$$

The backscattered SFCW frame in baseband is modeled by concatenating the down converted received pulses. The received pulse is an attenuated and delayed version of the transmitted pulse at a nominal distance R0. However, a slowly time-varying delay is expected due to target motion, R_{T}(t) is a function of slow-time t,

$${τ}_{D} ( t ) = 2 \frac{{R}_{0} + {R}_{T} ( t )}{c} \qquad Equation 13$$

where c denotes the speed of light. For example, the m-th frame of received waveform is written as,

xᵣₓ(t=mNT,τ=nT)=xᵣₓ(m,n) Equation 14

=e^{j2πƒ}^{₀}^{τ}^{_{D}}^{(m)}e^{-j2πnΔƒτ}^{_{D}}^{(m)} Equation 15

The range profile is obtained by performing inverse Fourier transform of the N fast frequency samples with respect to n for every frame. Then, the normalized baseband slow-time (m) versus fast-time (k) data matrix is computed as,

$${X}_{rx} ( m , k ) = {e}^{- j π ( k - {k}_{m} ) ( N - 1 ) / N} \frac{sin [ π ( k - {k}_{m} ) ]}{sin [ π ( k - {k}_{m} ) / N ]} {e}^{j 2 π {f}_{0} {τ}_{D} ( m )} \qquad Equation 16$$

The phase information directly related to motion of interest is preserved in the term e^{j2πƒ}^{₀}^{τ}^{_{D}}^{(m)}. To extract the signal of interest with maximum SNR, one fast-time delay sample (range sample) is selected across slow-time frames as k=kₘ, which is computed as the ceiling of τ_{D}(m)NΔƒ, since |Xᵣₓ(m,k)| achieves its maximum as k=kₘ.

C. Subjects and Experimental Protocol

Measurements were taken from 4 human test subjects. In total, the database contains 8540 seconds of data, comprising asynchronized raw data of PPG, rPPG and data derived from radar. All measurements were recorded under standardized conditions, seated comfortably in an armchair with back support and breathing normally at leisure. During the experiments, the distance between antenna and ROIs varied from 10 centimeters to 60 centimeters. Additionally, predefined interventions were considered, changing measurement positions including sitting, standing and lying down, changing heartbeat variability by physical exercising for 5 minutes before measurements, changing breathing pattern by holding breathing for 15 seconds to 30 seconds. Data acquisition was varied following the study protocol over different ROIs, such as finger, forehead, and inner elbow, which are illustrated in FIG. 13.

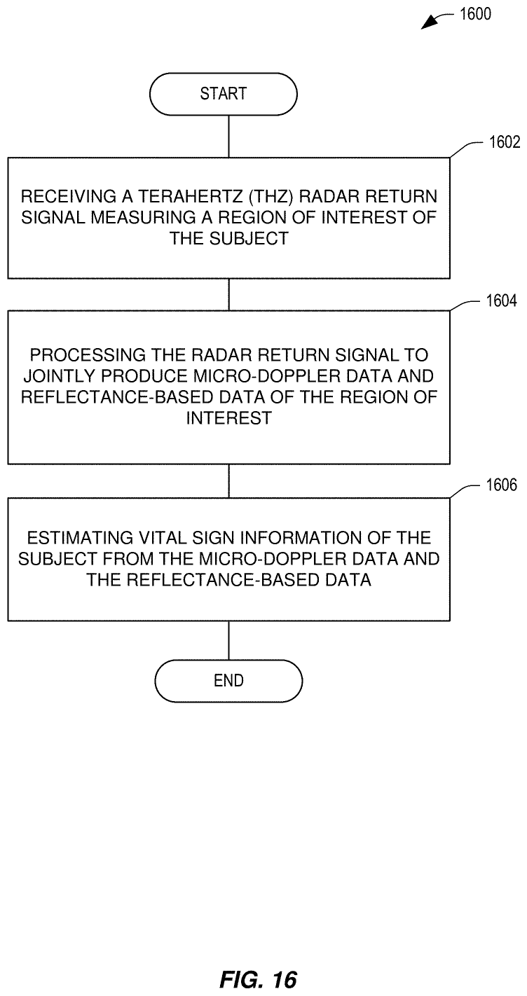

Turning now to FIG. 16, illustrated is an exemplary method 1600 for performing Terahertz-Wave-Plethysmography according to one or more embodiments disclosed herein.

The method 1600 can start at 1602 where the method includes receiving a terahertz (THz) radar return signal measuring a region of interest of the subject. The THz radar return signal is a return signal that was reflected off one or more subjects 108 in response to transmitting a 3D THz radar signal using either an UWB radar emitter or a stepped-frequency continuous wave radar emitter.

At 1604, the method includes processing (e.g., by the signal processor 102) the radar return signal that was reflected off the subject 108-1 and 108-2 to jointly produce micro-Doppler data and reflectance-based data of the region of interest.

At 1606, the method includes estimating (e.g., by the signal processor 102) vital sign information of the subject from the micro-Doppler data and the reflectance-based data. In some embodiments, the vital sign information can be based primarily on the reflectance-based data, with some small input from the micro-Doppler data.

### VII. Computer System

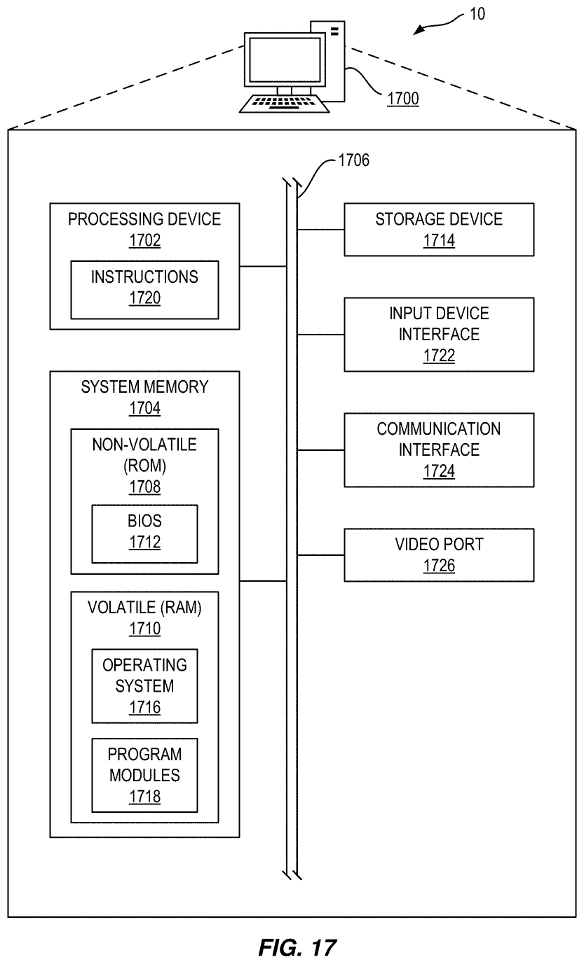

FIG. 17 is a block diagram of a TPG sensor 10 or other system or device (e.g., signal processor 102) suitable for implementing non-contact vital sign measurement of a subject according to embodiments disclosed herein. The TPG sensor 10 includes or is implemented as a computer system 1700, which comprises any computing or electronic device capable of including firmware, hardware, and/or executing software instructions that could be used to perform any of the methods or functions described above. In this regard, the computer system 1700 may be a circuit or circuits included in an electronic board card, such as a printed circuit board (PCB), a server, a personal computer, a desktop computer, a laptop computer, an array of computers, a personal digital assistant (PDA), a computing pad, a mobile device, or any other device, and may represent, for example, a server or a user's computer.

The exemplary computer system 1700 in this embodiment includes a processing device 1702 or processor, a system memory 1704, and a system bus 1706. The system memory 1704 may include non-volatile memory 1708 and volatile memory 1710. The non-volatile memory 1708 may include read-only memory (ROM), erasable programmable read-only memory (EPROM), electrically erasable programmable read-only memory (EEPROM), and the like. The volatile memory 1710 generally includes random-access memory (RAM) (e.g., dynamic random-access memory (DRAM), such as synchronous DRAM (SDRAM)). A basic input/output system (BIOS) 1712 may be stored in the non-volatile memory 1708 and can include the basic routines that help to transfer information between elements within the computer system 1700.

The system bus 1706 provides an interface for system components including, but not limited to, the system memory 1704 and the processing device 1702. The system bus 1706 may be any of several types of bus structures that may further interconnect to a memory bus (with or without a memory controller), a peripheral bus, and/or a local bus using any of a variety of commercially available bus architectures.

The processing device 1702 represents one or more commercially available or proprietary general-purpose processing devices, such as a microprocessor, central processing unit (CPU), or the like. More particularly, the processing device 1702 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 1702 is configured to execute processing logic instructions for performing the operations and steps discussed herein.

In this regard, the various illustrative logical blocks, modules, and circuits described in connection with the embodiments disclosed herein may be implemented or performed with the processing device 1702, which may be a microprocessor, field programmable gate array (FPGA), a digital signal processor (DSP), an application-specific integrated circuit (ASIC), or other programmable logic device, a discrete gate or transistor logic, discrete hardware components, or any combination thereof designed to perform the functions described herein. Furthermore, the processing device 1702 may be a microprocessor, or may be any conventional processor, controller, microcontroller, or state machine. The processing device 1702 may also be implemented as a combination of computing devices (e.g., a combination of a DSP and a microprocessor, a plurality of microprocessors, one or more microprocessors in conjunction with a DSP core, or any other such configuration).

The computer system 1700 may further include or be coupled to a non-transitory computer-readable storage medium, such as a storage device 1714, which may represent an internal or external hard disk drive (HDD), flash memory, or the like. The storage device 1714 and other drives associated with computer-readable media and computer-usable media may provide non-volatile storage of data, data structures, computer-executable instructions, and the like. Although the description of computer-readable media above refers to an HDD, it should be appreciated that other types of media that are readable by a computer, such as optical disks, magnetic cassettes, flash memory cards, cartridges, and the like, may also be used in the operating environment, and, further, that any such media may contain computer-executable instructions for performing novel methods of the disclosed embodiments.

An operating system 1716 and any number of program modules 1718 or other applications can be stored in the volatile memory 1710, wherein the program modules 1718 represent a wide array of computer-executable instructions corresponding to programs, applications, functions, and the like that may implement the functionality described herein in whole or in part, such as through instructions 1720 on the processing device 1702. The program modules 1718 may also reside on the storage mechanism provided by the storage device 1714. As such, all or a portion of the functionality described herein may be implemented as a computer program product stored on a transitory or non-transitory computer-usable or computer-readable storage medium, such as the storage device 1714, non-volatile memory 1708, volatile memory 1710, instructions 1720, and the like. The computer program product includes complex programming instructions, such as complex computer-readable program code, to cause the processing device 1702 to carry out the steps necessary to implement the functions described herein.

An operator, such as the user, may also be able to enter one or more configuration commands to the computer system 1700 through a keyboard, a pointing device such as a mouse, or a touch-sensitive surface, such as the display device, via an input device interface 1722 or remotely through a web interface, terminal program, or the like via a communication interface 1724. The communication interface 1724 may be wired or wireless and facilitate communications with any number of devices via a communications network in a direct or indirect fashion. An output device, such as a display device, can be coupled to the system bus 1706 and driven by a video port 1726. Additional inputs and outputs to the computer system 1700 may be provided through the system bus 1706 as appropriate to implement embodiments described herein.

The operational steps described in any of the exemplary embodiments herein are described to provide examples and discussion. The operations described may be performed in numerous different sequences other than the illustrated sequences. Furthermore, operations described in a single operational step may actually be performed in a number of different steps. Additionally, one or more operational steps discussed in the exemplary embodiments may be combined.

Those skilled in the art will recognize improvements and modifications to the preferred embodiments of the present disclosure. All such improvements and modifications are considered within the scope of the concepts disclosed herein and the claims that follow.

What is claimed is:

1. A method for non-contact vital sign measurement of a subject, the method comprising:
receiving a terahertz (THz) radar return signal measuring a region of interest of the subject;
processing the radar return signal to jointly produce micro-Doppler data and reflectance-based data of the region of interest; and
estimating vital sign information of the subject from the micro-Doppler data and the reflectance-based data.

2. The method of claim 1, wherein the radar return signal is received in response to a three-dimensional (3D) THz radar signal.

3. The method of claim 2, further comprising transmitting the 3D THz radar signal using an ultra-wideband (UWB) radar emitter.

4. The method of claim 3, wherein the 3D THz radar signal is between 100 gigahertz and 10 THz.

5. The method of claim 2, further comprising transmitting the 3D THz radar signal using a stepped-frequency continuous-wave (SFCW) radar emitter.

6. The method of claim 1, further comprising estimating a macro body motion of the subject using the micro-Doppler data.

7. The method of claim 1, further comprising extracting activity information from the micro-Doppler data.

8. The method of claim 7, wherein the activity information comprises at least one of a gait of the subject or a type of activity engaged in by the subject.

9. The method of claim 1, wherein the vital sign information comprises at least one of a heart rate, a heartbeat waveform, a heart rate variability (HRV), vascular aging information, or artery stiffness information.

10. The method of claim 1, wherein the micro-Doppler data is determined based on phase variation data associated with the radar return signal, and the reflectance-based data is based on magnitude variation data associated with the radar return signal.

11. A terahertz-wave-plethysmography (TPG) sensor, comprising:
a terahertz (THz) radar sensor; and
a signal processor configured to:
receive a radar return signal from the THz radar sensor;
measure a skin reflectance of the radar return signal; and
extract vital sign information of one or more subjects based on the skin reflectance.

12. The TPG sensor of claim 11, wherein the vital sign information comprises at least one of a heart rate, a heart signal, a heart rate variability (HRV), or inter-beat interval data of the one or more subjects.

13. The TPG sensor of claim 11, wherein the signal processor is further configured to acquire micro-Doppler data of a region of interest of the one or more subjects.

14. The TPG sensor of claim 11, wherein the signal processor is further configured to:
refine the vital sign information based on the micro-Doppler data.

15. The TPG sensor of claim 12, wherein the micro-Doppler data comprises a set of micro-Doppler images of the region of interest.

16. The TPG sensor of claim 11, wherein the radar return signal is reflected by a dermis layer of skin of the one or more subjects.

17. The TPG sensor of claim 11, further comprising:
an ultra-wideband (UWB) radar emitter that emits a three-dimensional (3D) THz radar signal, wherein the radar return signal is a reflection of the 3D THz radar signal.

18. The TPG sensor of claim 11, further comprising:
a stepped-frequency continuous-wave (SFCW) radar emitter that emits a three-dimensional (3D) THz radar signal, wherein the radar return signal is a reflection of the 3D THz radar signal.

19. The TPG sensor of claim 10, wherein the signal processor is further configured to identify a first human subject and a second human subject based on the radar return signal.

20. A non-transitory computer-readable medium comprising computer-readable instructions, that in response to being executed by a processor, cause the processor to:
receive a radar return signal from a terahertz (THz) radar sensor;
measure a skin reflectance of the radar return signal; and
extract vital sign information of one or more subjects based on the skin reflectance.
